(19) United States
(12) Patent Application Publication
(10) Pub. No.: US 2024/0424275 A1
(43) Pub. Date: Dec. 26, 2024

(54) TRANSFER INSTRUMENT

(71) Applicant: TERUMO KABUSHIKI KAISHA, Tokyo (JP)

(72) Inventor: Koichi HAYAKAWA, Kanagawa (JP)

(73) Assignee: TERUMO KABUSHIKI KAISHA, Tokyo (JP)

(21) Appl. No.: 18/741,278

(22) Filed: Jun. 12, 2024

(30) Foreign Application Priority Data
Jun. 20, 2023 (JP) 2023-100693

(51) Int. Cl. A61M 37/00

(52) U.S. Cl. CPC A61M 37/0069; A61M 2202/0007; A61M 2202/06; A61M 2205/0216; A61M 2210/125

(57) ABSTRACT

A transfer instrument is provided with a carrier holding portion at a distal end of a second shaft. The carrier holding portion includes a pressing surface and a first projection. The pressing surface presses a medical sheet supported on a first support surface of a first support portion in a distal end direction. The first projection is provided remote from the first support surface relative to the pressing surface and protrudes in the distal end direction relative to the pressing surface.

### CROSS-REFERENCES TO RELATED APPLICATIONS

This application is based on and claims priority to Japanese Patent Application No. 2023-100693 filed on Jun. 20, 2023, the entire content of which is incorporated herein by reference.

### TECHNOLOGICAL FIELD

The present invention generally relates to a transfer instrument for transferring a medical sheet to a treatment site of a living body.

### BACKGROUND DISCUSSION

Japanese Patent Application Publication No. 2009-000511 (JP 2009-000511 A) discloses a transfer instrument for transferring a medical sheet (cell sheet) for use in, for example, organ transplantation to a treatment site of a living body. The transfer instrument includes an outer cylinder, a slide member slidably supported in the outer cylinder, and a seat support member provided at a distal end of the slide member.

### SUMMARY

It is desired to transfer a medical sheet to a treatment site more efficiently.
(1) A transfer instrument used to transfer a medical sheet to a treatment site of a living body, the transfer instrument including: a first carrier member including a first shaft and a first support portion, the first support portion being provided at a distal end of the first shaft and having a first support surface that is configured to support the medical sheet; and a second carrier member including a second shaft and a pressing portion, the second shaft extending along the first shaft and being movable along the first shaft, the pressing portion being provided at a distal end of the second shaft, in which the pressing portion includes a pressing surface and a projection, the pressing surface being configured to move the medical sheet in a distal end direction when the medical sheet is supported on the first support surface, the projection being provided spaced from the first support surface relative to the pressing surface and protruding in the distal end direction relative to the pressing surface.

With this configuration, it is possible to efficiently transfer, by moving the second shaft along the first shaft with the medical sheet supported on the first support surface, the medical sheet to the treatment site.

Further, with this configuration, the projection protruding in the distal end direction relative to the pressing surface is provided remote from the first support surface relative to the pressing surface. Accordingly, when the second shaft is moved in the distal end direction to cause the pressing surface to press the medical sheet in the distal end direction, the projection is positioned above the medical sheet, so that it is possible to prevent the medical sheet from running on to the pressing portion. As a result, the medical sheet can be smoothly transferred to the treatment site.
(2) The transfer instrument according to the above (1), in which the second carrier member may further include a second support portion fixed to the pressing portion and having a second support surface smaller than the first support surface, the medical sheet may be supported on the second support surface, and the pressing surface, the projection, and the second support portion may form a first recess that a proximal end of the medical sheet enters.

With this configuration, the second support portion is fixed to the pressing surface, and the medical sheet is supported on the second support surface. Accordingly, when the second shaft is moved in the distal end direction, the proximal end of the medical sheet enters the first recess to come into contact with the pressing surface. As a result, it is possible to smoothly transfer, by pressing the medical sheet in the distal end direction with the pressing surface, the medical sheet to the treatment site while reliably preventing the medical sheet from running on to the pressing portion.
(3) The transfer instrument according to the above (2), in which the pressing portion may further include a second projection provided adjacent to the first support surface relative to the pressing surface and protruding in the distal end direction relative to the pressing surface, and the pressing surface, the second projection, and the second support portion may form a second recess that the proximal end of the medical sheet enters.

With this configuration, the second projection is provided adjacent to the first support surface relative to the pressing surface. Accordingly, when the medical sheet is placed on the second support portion with the second shaft rotated by 180° about the axis of the second shaft, the second projection is positioned below the medical sheet. Even in a case where the second shaft rotates about the axis of the second shaft, it is possible to cause, by moving the second shaft in the distal end direction, the proximal end of the medical sheet to enter the second recess to come into contact with the pressing surface. As a result, it is possible to smoothly transfer, by pressing the medical sheet in the distal end direction with the pressing surface, the medical sheet to the treatment site while reliably preventing the medical sheet from running on to the pressing portion.
(4) The transfer instrument according to any one of the above (1) to (3), in which a protrusion length of the projection in the distal end direction may be greater than or equal to half of a total length of the medical sheet in the distal end direction.

With this configuration, since the protrusion length of the projection in the distal end direction is greater than or equal to half of the total length of the medical sheet, the medical sheet can be reliably supported between the first support portion and the projection. Further, when the pressing surface presses the medical sheet in the distal end direction, it is possible to prevent the medical sheet from becoming separated upward from the first support surface and crinkled.
(5) The transfer instrument according to the above (4), in which the pressing portion may include a flexible material, the projection may include a lower surface facing the first support surface and an upper surface opposite to the lower surface, and on the upper surface, a rib protruding away from the first support surface and extending in the distal end direction may be provided.

With this configuration, the pressing portion provided with the projection includes a flexible material, and the rib is provided on the upper surface of the projection. It is therefore possible to easily place the medical sheet between the first support portion and the projection with the rib lifted.
(6) The transfer instrument according to any one of the above (1) to (5), in which the projection may have a distal end formed in an arc shape.

With this configuration, since the projection has its distal end formed in an arc shape, it is possible to prevent the medical sheet from breaking when the medical sheet comes into contact with the distal end of the projection.

The transfer instrument makes it possible to efficiently transfer, by moving the second shaft along the first shaft with the medical sheet supported on the first support surface, the medical sheet to the treatment site.

Further, the projection protruding in the distal end direction relative to the pressing surface is provided remote from the first support surface relative to the pressing surface. Accordingly, when the second shaft is moved in the distal end direction to cause the pressing surface to press the medical sheet in the distal end direction, the projection is positioned above the medical sheet, so that it is possible to prevent the medical sheet from running on to the pressing portion. As a result, the medical sheet can be smoothly transferred to the treatment site.

According to another aspect, a transfer instrument for transferring a medical sheet to a treatment site of a living body comprises: an outer cylinder having a through hole that extends throughout the outer cylinder from a distal-end opening at a distal end of the outer cylinder to a proximal-end opening at a proximal end of the outer cylinder, a first carrier member that includes a first shaft and a first support portion, and a second carrier member that includes a second shaft and a pressing portion. The first shaft has a distal end portion at which is fixed the first support portion so that the first shaft and the first support portion move together, with the first support portion extending in a distal direction away from the first shaft and having a first support surface configured to support the medical sheet. The first shaft is positionable in the outer cylinder so that a proximal portion of the first shaft is proximal of the proximal-end opening of the outer cylinder while the first support portion is positioned inside the outer cylinder. The first shaft is axially movable in a distal direction relative to the outer cylinder to move the first support portion from inside the outer cylinder to an extended position in which the first support portion is distal of the distal-end opening of the outer cylinder, with the first shaft having a lumen that extends throughout the first shaft so that the lumen opens to a distal end of the first shaft and opens to a proximal end of the first shaft. The second shaft has a distal end portion at which is fixed the pressing portion so that the second shaft and the pressing portion move together. The second shaft is positionable in the lumen of the first shaft with a proximal portion of the second shaft positioned proximal of the proximal end of the first shaft and the pressing portion positioned in the lumen of the first shaft. The pressing portion has a pressing surface facing in the distal direction, with the pressing portion including a projection that extends in the distal direction and that extends distally beyond the pressing surface. The second shaft, when positioned in the lumen of the first shaft, is axially movable in the distal direction relative to the first shaft when the first shaft is positioned in the outer cylinder with the first support portion in the extended position and the medical sheet supported on the first support portion so that the axial movement of the second shaft in the distal direction relative to the first shaft causes the pressing surface to contact a periphery of the medical device supported on the first support portion and move the medical device in the distal direction while the projection is positioned in overlying relation to the medical sheet and a lower surface of the projection faces toward and is spaced from the medical sheet.

Another aspect involves a method comprising positioning an outer cylinder, a first carrier member and a second carrier member adjacent a site. The first carrier member includes a first shaft, a first support portion fixed to the first shaft and positioned in the outer cylinder, and a medical sheet surrounded by the first support portion. The second carrier member includes a second shaft and a pressing portion fixed to the second shaft and positioned in the outer cylinder, with the pressing portion including a pressing surface and a projection. The method also includes axially moving the first shaft relative to the outer cylinder to move the first support portion to a position distal of a distal end of the outer cylinder so that the medical sheet is supported by the support portion and is positioned outside the outer cylinder, axially moving the second shaft relative to the first shaft to move the pressing surface into contact with a periphery of the medical sheet, and further axially moving the second shaft relative to the first shaft to move the pressing surface that is in contact with the periphery of the medical sheet in a distal direction to move the medical sheet in the distal direction relative to the first support portion.

### BRIEF DESCRIPTION OF THE DRAWINGS

FIG. 1 is a perspective view of a transfer instrument according to a first embodiment.

FIG. 2 is an exploded perspective view of the transfer instrument illustrated in FIG. 1.

FIG. 3 is a plan view of a distal end of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a flowchart illustrating a procedure of a transfer method for transferring a medical sheet using the transfer instrument illustrated in FIG. 1.

FIG. 7 is a first explanatory diagram illustrating how the medical sheet is placed in a sheet placing process.

FIG. 8 is a second explanatory diagram illustrating how the medical sheet is placed in the sheet placing process.

FIG. 9 is an explanatory diagram of a retracting process.

FIG. 10 is a transverse cross-sectional view taken along line X-X in FIG. 9.

FIG. 11 is an explanatory diagram of a positioning process.

FIG. 12 is an explanatory diagram of an unfolding process.

FIG. 13 is a first explanatory diagram of a moving process.

FIG. 14 is a second explanatory diagram of the moving process.

FIG. 15 is a third explanatory diagram of the moving process.

FIG. 16 is an explanatory diagram of a withdrawing process.

FIG. 17 is a perspective view of a transfer instrument according to a second embodiment.

FIG. 18 is an explanatory diagram of a retracting process.

FIG. 19 is a transverse cross-sectional view taken along line XIX-XIX in FIG. 18.

FIG. 20 is an explanatory diagram of an unfolding process.

FIG. 21 is a first explanatory diagram of a moving process.

FIG. 22 is a second explanatory diagram of the moving process.

FIG. 23 is a third explanatory diagram of the moving process.

### DETAILED DESCRIPTION

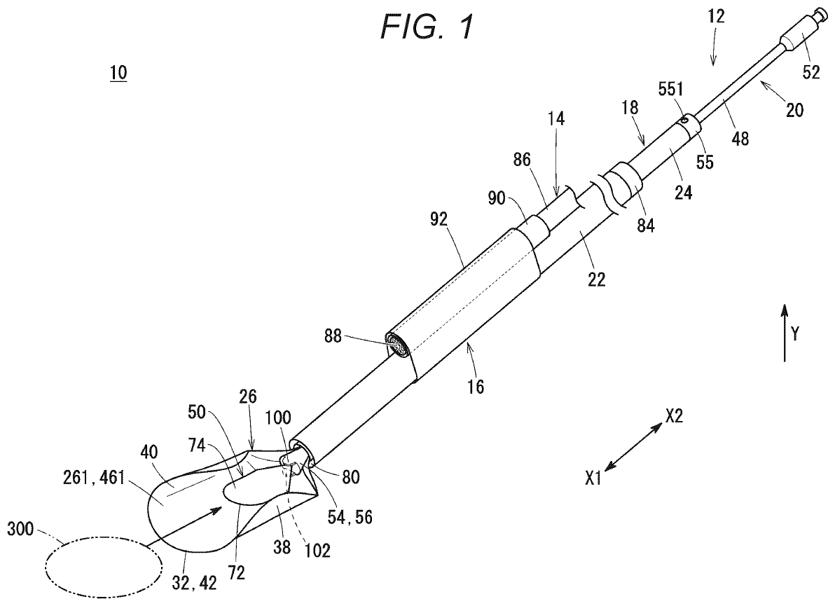

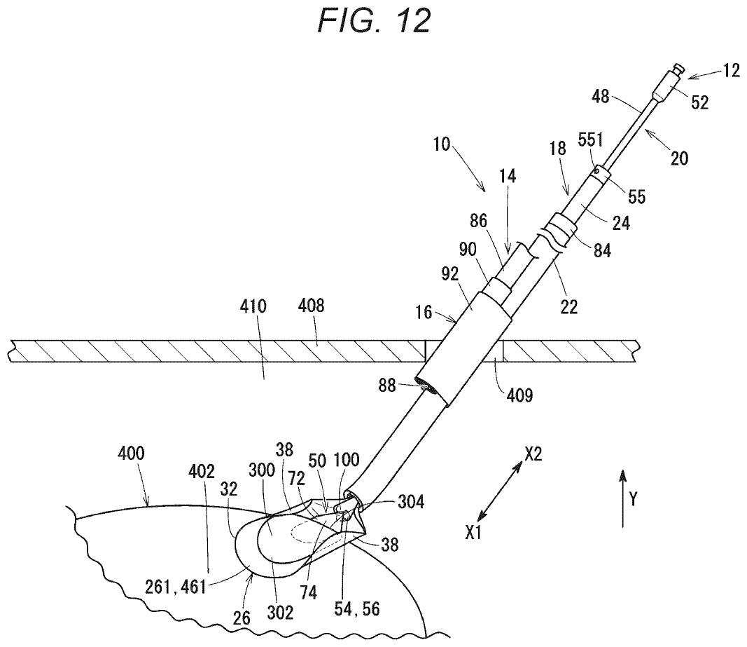

FIG. 1 is a perspective view of a transfer instrument 10 according to a first embodiment. The transfer instrument 10 is a medical instrument for transferring a medical sheet 300 to a treatment site of a living body. The transfer instrument 10 is used for, for example, the treatment of severe heart failure caused by ischemic heart disease. In this case, the medical sheet 300 is transplanted to a recipient site 402 of a heart 400 (the treatment site of the living body) (see FIGS. 12 to 16). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. The thickness and the diameter (size) of the medical sheet 300 are not limited these values and can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

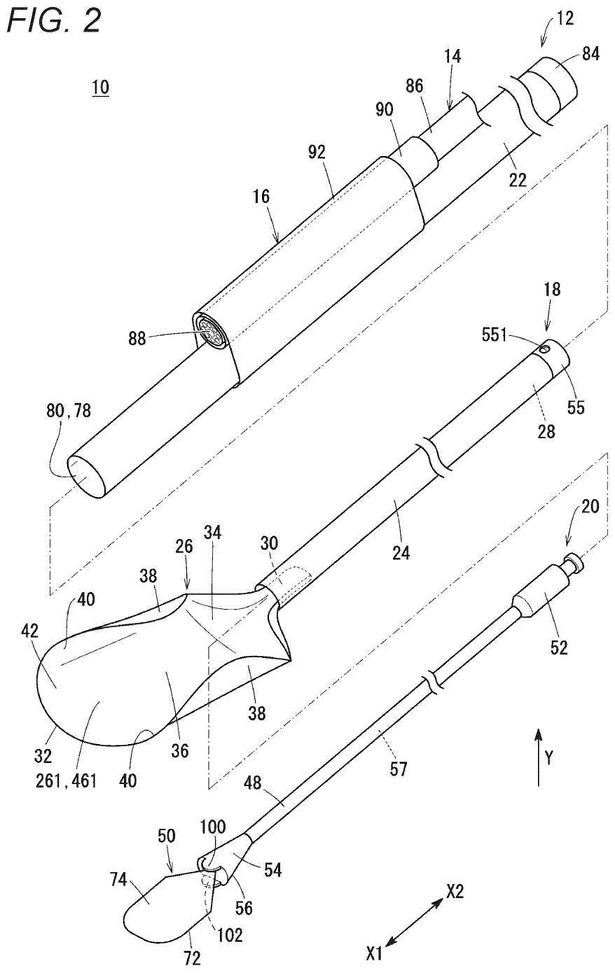

As illustrated in FIGS. 1 and 2, the transfer instrument 10 includes an instrument body 12, an endoscope 14, and a fixing member 16. The instrument body 12 includes a first carrier member 18, a second carrier member 20, and an outer cylinder 22. The transfer instrument 10 is not limited to a configuration including the endoscope 14.

In FIG. 2, the first carrier member 18 includes a first shaft 24 and a first support portion 26.

The first shaft 24 is a tubular body (in the first embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 has an airtight valve 55 provided at its proximal end with the valve 55 in close contact with an outer circumferential surface of the second shaft 48. The valve 55 has a marker 551 provided on its outer circumferential surface. When the transfer instrument 10 is used, the marker 551 is visible to the user. Note that the first shaft 24 is not limited to a tubular body and may be a body other than a tubular body.

The first shaft 24 extends in an axial direction of the outer cylinder 22 and is disposed inside the outer cylinder 22 in a manner as to be movable along the axial direction. The first shaft 24 includes, for example, a resin material. Examples of the constituent material from which the first shaft 24 may be fabricated include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and can maintain the bent shape.

As illustrated in FIGS. 2 to 4, the first support portion 26 is attached to the distal end of the first shaft 24. The first support portion 26 includes or is fabricated from, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material from which the first support portion 26 may be fabricated preferably has transparency, and examples of the constituent material include, but not particularly limited to, polyethylene, polycarbonate, polyamide, polystyrene, polypropylene, polyacetal resin, polyimide, polyetherimide, polyetheretherketone, polyethylene terephthalate, and fluororesin. Further, the first support portion 26 may have a mesh shape.

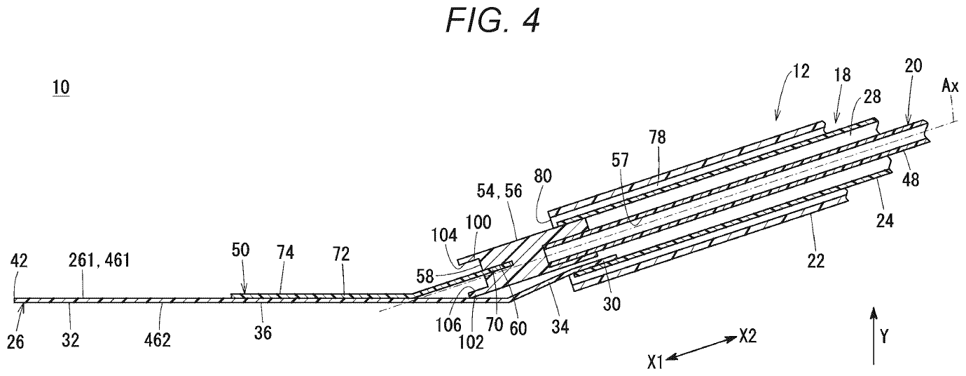

In FIG. 4, the first joint 30 is bonded to an inner circumferential surface of the distal end of the first shaft 24 with an adhesive. Examples of the adhesive include, but not particularly limited to, a UV adhesive, a hot-melt adhesive, and an instant adhesive (for example, cyanoacrylate-based instant adhesive). The first joint 30 may be thermally fused to the inner circumferential surface of the first shaft 24. Note that the first support portion 26 may be detachable from the distal end of the first shaft 24.

As illustrated in FIG. 2, the first support body 32 extends in a distal end direction from first joint 30. The first support body 32 includes a proximal-end support portion 34, an intermediate support portion 36, a pair of first protrusions 38, a pair of second protrusions 40, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26.

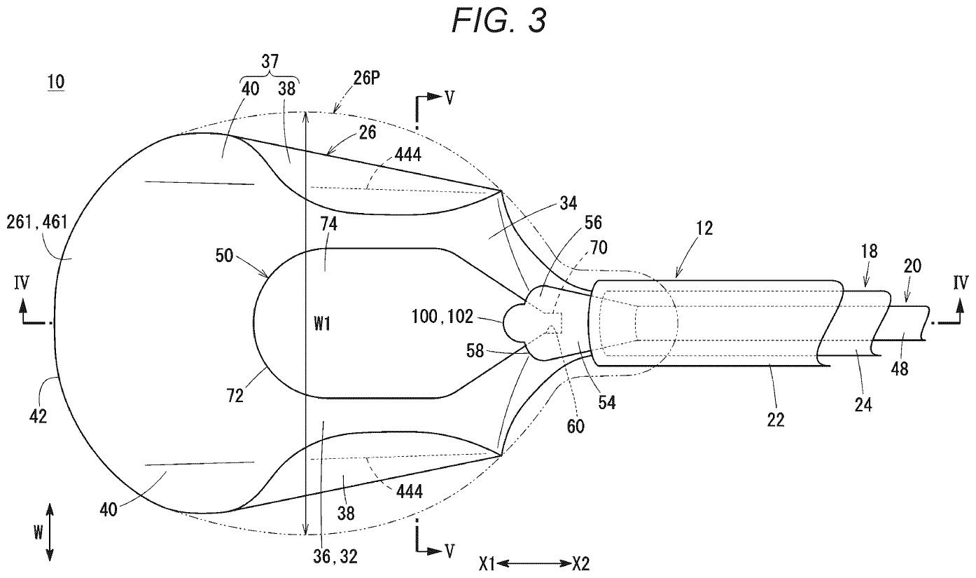

As illustrated in FIG. 3, the proximal-end support portion 34 is formed to be wider in its extending direction. In other words, both sides of the proximal-end support portion 34 in a width direction are tapered toward the first joint 30 (see FIG. 2). The intermediate support portion 36 is formed in a tapered shape so as to be gradually narrower in width from the distal end toward the proximal end (in the direction of arrow X2).

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction perpendicular to the first support surface 261 illustrated in FIG. 3, the distal-end support portion 42 of the first support portion 26 has an arc shape connecting the pair of second protrusions 40.

As illustrated in FIG. 4, the proximal-end support portion 34 extends roughly along an axis Ax of the first shaft 24 from a distal end of the first joint 30 in the distal end direction (the direction of arrow X1) (see FIG. 4). The intermediate support portion 36 intersects with the axis Ax of the first shaft 24 and extends from a distal end of the proximal-end support portion 34 toward the distal end of the first support portion 26 (in the direction of arrow X1).

In FIGS. 2 and 3, the pair of first protrusions 38 protrude upward (in the direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34. Each first protrusion 38 has a convex shape extending in a direction away from the first support surface 261.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

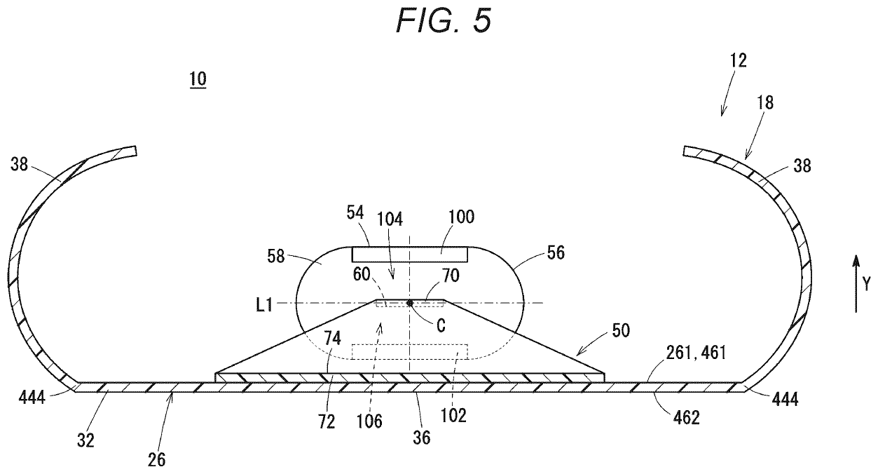

Each of the pair of first protrusions 38 has one of a pair of bent portions 444. Each of the pair of bent portions 444 causes a corresponding one of the pair of first protrusions 38 to bend from the first support surface 261 (intermediate support portion 36) of the first support portion 26 (see FIG. 5).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52.

The second shaft 48 is a tubular body (in the first embodiment, a circular tube member) having a second lumen 57. The second shaft 48 is longer in the axial direction than the first shaft 24. The second shaft 48 is inserted into or positioned in the first lumen 28 of the first shaft 24 (see FIGS. 1 and 4). In other words, a distal end of the second shaft 48 protrudes in the distal end direction (the direction of arrow X1) relative to the distal-end opening of the first shaft 24. A proximal end of the second shaft 48 protrudes in the proximal end direction (the direction of arrow X2) relative to the proximal-end opening of the first shaft 24 (see FIG. 1). The second shaft 48 is provided extending along the first shaft 24 so as to be movable along the first shaft 24. The second shaft 48 is not limited to a tubular body and may be a body other than a tubular body.

The second shaft 48 is configured to follow the shape of the first support portion 26. As the constituent material of the second shaft 48 may be fabricated, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 (pressing portion) and a pressure-application portion 56 that is the distal end of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the first embodiment, the pressure-application portion 56 is provided with the carrier holding portion 54 that supports the second support portion 50. The carrier holding portion 54 includes the pressing surface 58 and an attachment hole 60.

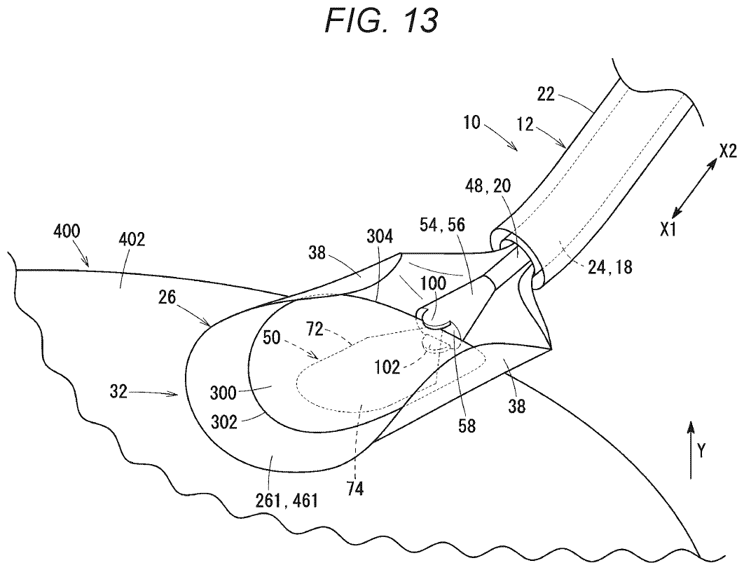

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIGS. 13 to 15).

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into or positioned in the attachment hole 60 of the carrier holding portion 54, and is bonded, for example. The second joint 70 may be joined to the attachment hole 60 of the carrier holding portion 54 by a suitable joining method other than adhesion. The second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided, on its upper surface, with a second support surface 74 on which the medical sheet 300 is placed. The second support surface 74 includes a flat surface. The second support body 72 is smaller than the first support body 32. That is, the second support surface 74 is smaller in area than the first support surface 261.

In FIG. 2, the hub 52 is attached to the proximal end of the second shaft 48.

In FIGS. 1 and 2, the outer cylinder 22 is a cylindrical member having a lumen 78. The lumen 78 has a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material from which the outer cylinder 22 may be fabricated are the same as the examples of the constituent material of the first shaft 24 described above.

The first shaft 24 is inserted into or positioned in the lumen 78 of the outer cylinder 22. The outer cylinder 22 is shorter in length along the axial direction than the first shaft 24. The outer cylinder 22 is provided with, at its proximal end, an airtight valve 84 that is in close contact with an outer circumferential surface of the first shaft 24.

In FIGS. 2 and 4, a distal end surface of the outer cylinder 22 extends orthogonal to the axial direction of the outer cylinder 22.

As illustrated in FIG. 2, the endoscope 14 includes a long or elongated endoscope main body 86. A distal end of the endoscope main body 86 is fixed to an outer circumferential surface of the outer cylinder 22 by the fixing member 16 (see FIG. 1). An objective lens 88 provided on a distal end surface of the endoscope main body 86 is oriented toward the distal end of the outer cylinder 22 (in the direction of arrow X1). The distal end of the endoscope main body 86 is fixed to an intermediate portion of the outer cylinder 22 in the axial direction. The distal end of the endoscope main body 86 may be fixed to the distal end of the outer cylinder 22.

The fixing member 16 includes, for example, a fixing cylinder 90 and a fixing tube 92. The fixing cylinder 90 includes, for example, a hard resin material. The endoscope main body 86 can be inserted into or positioned in a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 is, for example, a heat-shrink tube. The manner in which the distal end of the endoscope main body 86 is fixed to the outer cylinder 22 may be determined as desired.

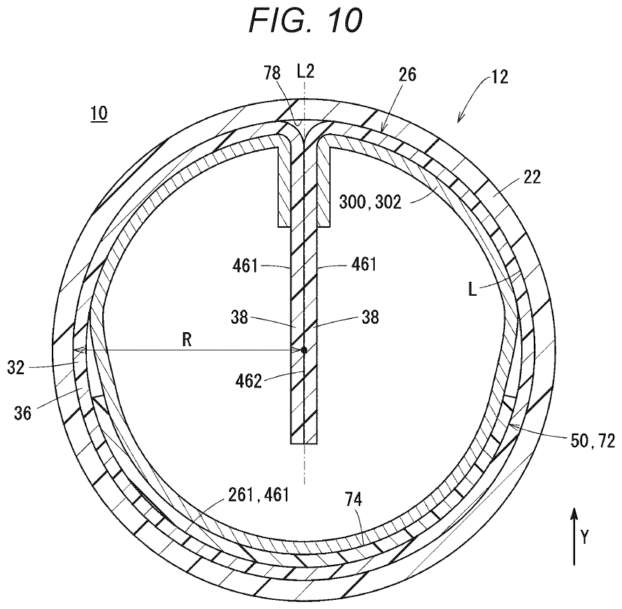

As illustrated in FIG. 3, a portion including both the first protrusion 38 and the second protrusion 40 of the first support portion 26 is hereinafter referred to as “protrusion 37”. Therefore, the protrusion 37 is provided as a pair on both sides of the first support portion 26 in the width direction. In FIG. 3, the direction (W direction) orthogonal to the axial direction of the first shaft 24 refers to the width direction of the first support portion 26. The W direction also refers to a width direction of other components (the outer cylinder 22 and the like). In the first embodiment, the first support portion 26 includes the pair of protrusions 37, and W1 in FIG. 3 denotes a width of the widest portion of the first support portion 26 when the first support portion 26 is unfolded to be flat. That is, the width W1 of the first support portion 26 is the maximum width of a first support portion 26P in a flat state before the pair of protrusions 37 are formed (before folded). The width W1 of the first support portion 26 is larger than an inner circumferential length L of the outer cylinder 22 (see FIG. 10). In FIG. 10, the inner circumferential length L of the outer cylinder 22 is a length of an inner circumferential surface along a circumferential direction of the outer cylinder 22 (L=2πR). R denotes the radius of the lumen 78 of the outer cylinder 22.

As illustrated in FIG. 5, in the cross section orthogonal to the axis of the second shaft 48, the carrier holding portion 54 has a line-symmetrical shape with respect to an imaginary line L1 passing through a center line C of the carrier holding portion 54 and parallel to the width direction of the carrier holding portion 54. In other words, the carrier holding portion 54 has a symmetrical shape in a vertical direction with respect to the imaginary line L1. As illustrated in FIG. 5, the carrier holding portion 54 has a flattened cross-sectional shape. The carrier holding portion 54 is not limited to a configuration formed in a flattened cross-sectional shape, and may be formed in, for example, a circular cross-sectional shape, an elliptical cross-sectional shape, a square cross-sectional shape, a rectangular cross-sectional shape, or the like.

The attachment hole 60 is disposed on the center line C of the carrier holding portion 54 on the pressing surface 58. The attachment hole 60 has a slit shape extending parallel to the width direction of the carrier holding portion 54 from the center line C. The attachment hole 60 has a symmetrical shape in the width direction with respect to the center line C. The attachment hole 60 extends in the axial direction from the pressing surface 58 (see FIG. 4). As described above, the second joint 70 of the second support portion 50 is inserted into and connected to the attachment hole 60.

In FIGS. 1 to 5, the carrier holding portion 54 further includes a first projection 100 (projection) and a second projection 102 (second projection). The first projection 100 and the second projection 102 are provided at the distal end of the carrier holding portion 54. The first projection 100 and the second projection 102 have the same shape. The first projection 100 and the second projection 102 may have different shapes.

Specifically, as illustrated in FIGS. 4 and 5, the first projection 100 is provided remote from the first support surface 261 relative to the pressing surface 58 at the distal end of the carrier holding portion 54. That is, as shown, the first projection 100 is spaced from the first support surface 261 such that the distance from the first support surface 261 to the first projection 100 is greater than the distance from the first support surface 261 to the pressing surface 58. That is, the first projection 100 is provided above the pressing surface 58 at the distal end of the carrier holding portion 54. As illustrated in FIGS. 2 to 4, the first projection 100 protrudes in the distal end direction relative to the pressing surface 58 at the distal end of the carrier holding portion 54. As illustrated in FIGS. 2 and 3, the first projection 100 protrudes in a semicircular shape from the distal end of the carrier holding portion 54.

The first projection 100 has a distal end formed in an arc shape. The shape of the distal end of the first projection 100 is not limited to the arc shape. The distal end of the first projection 100 may have any shape as long as the medical sheet 300 does not break when the distal end comes into contact with the medical sheet 300.

Specifically, as illustrated in FIGS. 4 and 5, the second projection 102 is provided adjacent to the first support surface 261 relative to the pressing surface 58 at the distal end of the carrier holding portion 54. That is, the second projection 102 is provided below the pressing surface 58 at the distal end of the carrier holding portion 54. As illustrated in FIGS. 2 to 4, the second projection 102 protrudes in the distal end direction relative to the pressing surface 58 at the distal end of the carrier holding portion 54. As illustrated in FIG. 2, the second projection 102 protrudes in a semicircular shape from the distal end of the carrier holding portion 54.

The second projection 102 has a distal end formed in an arc shape. The shape of the distal end of the second projection 102 is not limited to the arc shape. The distal end of the second projection 102 may have any shape as long as the medical sheet 300 does not break when the distal end comes into contact with the medical sheet 300.

As described above, the carrier holding portion 54 is symmetrical in the vertical direction with respect to the imaginary line L1. Therefore, the first projection 100 and the second projection 102 are provided at equal intervals in the vertical direction from the center line C.

As illustrated in FIG. 4, the first projection 100 and the second projection 102 have the same protrusion amount in the distal end direction. As illustrated in FIG. 5, the first projection 100 and the second projection 102 have the same width in the width direction of the carrier holding portion 54.

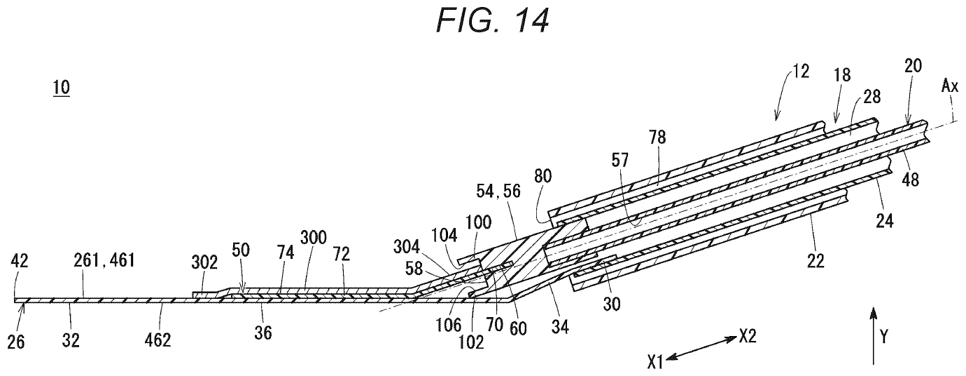

As illustrated in FIGS. 4 and 5, a portion of the pressing surface 58 above the second support portion 50, the first projection 100, and the second support portion 50 form a first recess 104. When the medical sheet 300 is placed on the second support surface 74, a proximal end 304 of the medical sheet 300 can enter the first recess 104 (see FIGS. 13 and 14).

As illustrated in FIGS. 4 and 5, a portion of the pressing surface 58 below the second support portion 50, the second projection 102, and the second support portion 50 form a second recess 106. As described above, the carrier holding portion 54 has a symmetrical shape in the vertical direction with respect to the imaginary line L1 (see FIG. 5). Therefore, in a case where the user rotates the second shaft 48 about the axis of the second shaft 48 to invert the second support portion 50 vertically, the second projection 102 is positioned at an upper part of the carrier holding portion 54. At this time, if the medical sheet 300 is placed on the second support portion 50, the proximal end 304 of the medical sheet 300 can enter the second recess 106.

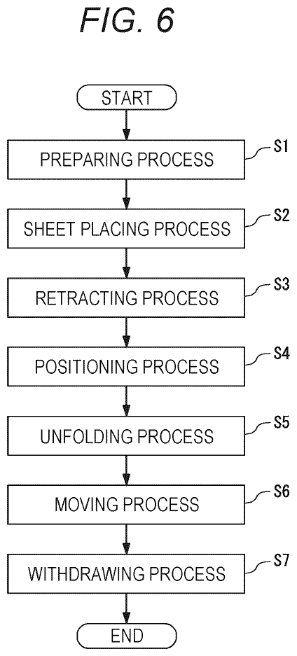

Next, a transfer method for transferring the medical sheet 300 to a treatment site of a living body will be described. Specifically, as illustrated in FIGS. 12 to 16, a transfer method for transferring the medical sheet 300 to the recipient site 402 of the heart 400 (the treatment site of the living body) during thoracoscopic surgery will be described. As illustrated in FIG. 6, the transfer method according to the first embodiment includes a preparing process, a sheet placing process, a retracting process, a positioning process, an unfolding process, a moving process, and a withdrawing process.

First, in the preparing process (step S1), the transfer instrument 10 according to the first embodiment described above is prepared. The following description will be given on the assumption that a state as illustrated in FIG. 1 is an initial state of the transfer instrument 10. In the initial state, the first shaft 24 and the second shaft 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, a proximal end of the carrier holding portion 54 is in the first lumen 28 of the first shaft 24.

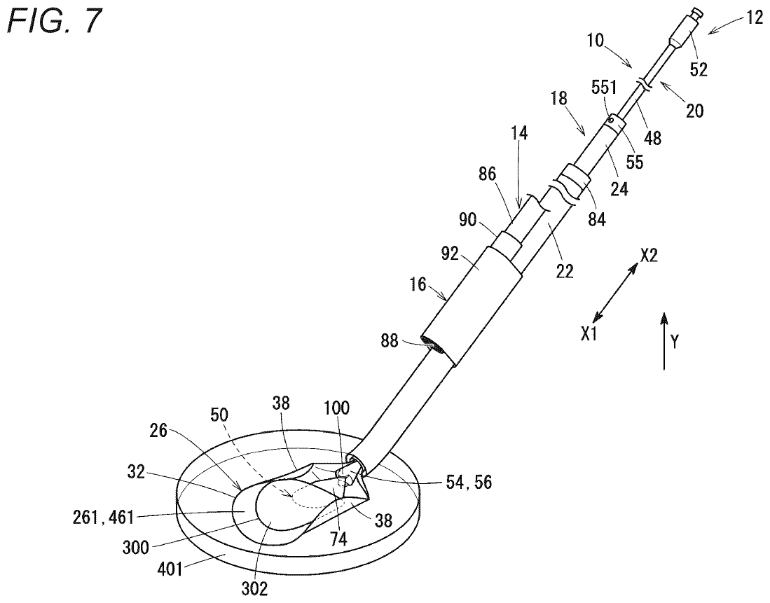

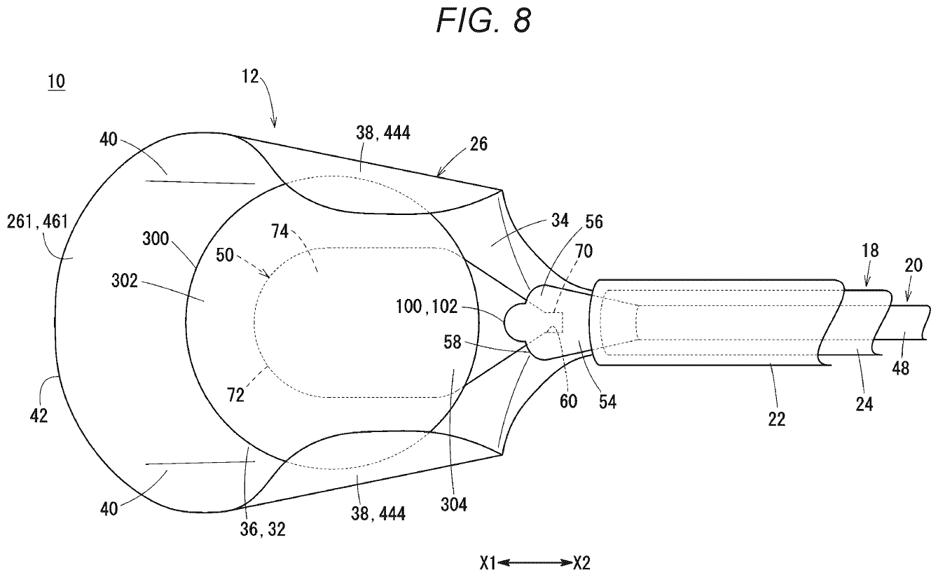

Next, in the sheet placing process (step S2), as illustrated in FIGS. 7 and 8, the medical sheet 300 placed on a Petri dish 401 is placed on the second support surface 74. The medical sheet 300 protrudes outward from the second support portion 50 with the medical sheet 300 placed on the second support surface 74. The first support surface 261 supports an overhanging portion 302 of the medical sheet 300 that sticks out over the second support portion 50.

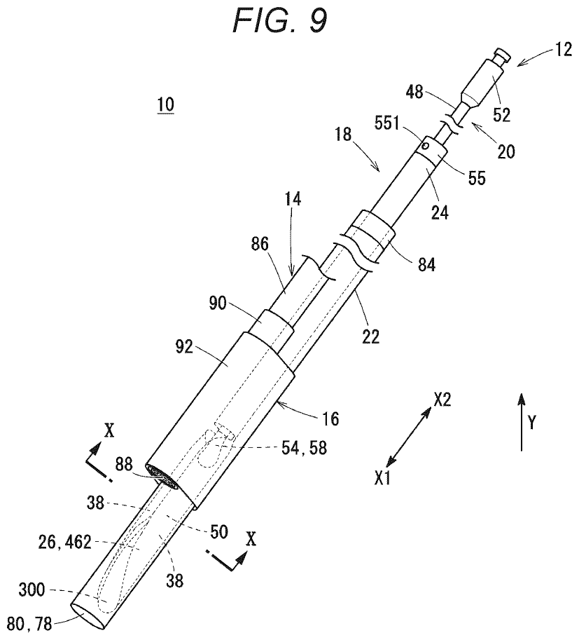

Subsequently, in the retracting process (step S3 in FIG. 6), as illustrated in FIG. 9, the medical sheet 300 is retracted in the outer cylinder 22 together with the first support portion 26 and the second support portion 50 (see FIG. 8) at a retracted position (first position). Specifically, the first shaft 24 of the first carrier member 18 and the second shaft 48 of the second carrier member 20 are moved together in the proximal end direction (the direction of arrow X2) relative to the outer cylinder 22.

Then, the proximal-end support portion 34 (see FIG. 2) of the first support portion 26 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to cause the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted in the outer cylinder 22 while curling in a conical shape such that the distal end side of the first support portion 26 becomes larger in diameter than the proximal-end support portion 34.

When the proximal-end support portion 34 of the first support portion 26 becomes deformed, a force acts on the intermediate support portion 36 of the first support portion 26 to cause the intermediate support portion 36 to curl along the circumferential direction of the outer cylinder 22, so that the intermediate support portion 36 (see FIG. 2) is pulled into the outer cylinder 22 while curling. At this time, the intermediate support portion 36 becomes deformed into a cylindrical shape along the inner surface of the outer cylinder 22. Each of the pair of first protrusions 38 is curved such that the front surface 461 of the first support portion 26 faces inward and the back surface 462 of the first support portion 26 faces outward. As illustrated in FIG. 10, the back surfaces 462 of both sides of the first support portion 26 in the width direction come into contact with each other on an imaginary line L2 extending orthogonal to the center axis of the outer cylinder 22. One of the first protrusions 38 and the other first protrusion 38 come into contact with each other and are retracted downward (toward the first support surface 261, the front surface 461).

Accordingly, the back surface 462 of the first support portion 26 is curved and is in close contact with the inner surface of the outer cylinder 22, each of the first protrusions 38 is further curved to fold back toward the center of the outer cylinder 22, and a free end of each of the first protrusions 38 is positioned below the center axis of the outer cylinder 22. That is, the first support portion 26 is curved in a heart shape along the inner surface of the outer cylinder 22.

The heart shape refers to a roughly round shape including two convex shapes, a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other so as to bring their respective circumferential surfaces into partial contact with each other, so that the entire contour becomes a roughly round shape along the inner surface of the tubular body (refer to the shape of the first support portion 26 in FIG. 10).

Along with the curved deformation of the first support portion 26, the second support portion 50 is similarly curved and deformed along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted in the outer cylinder 22 accordingly.

As illustrated in FIG. 9, when the first support portion 26 is entirely inserted into the outer cylinder 22, the retracting process is complete. At the completion of the retracting process, the first support portion 26 may partially protrude from the distal-end opening 80 of the outer cylinder 22. In this case, the state where the first support portion 26 partially protrudes from the distal-end opening 80 of the outer cylinder 22 corresponds to the first position of the first support portion 26.

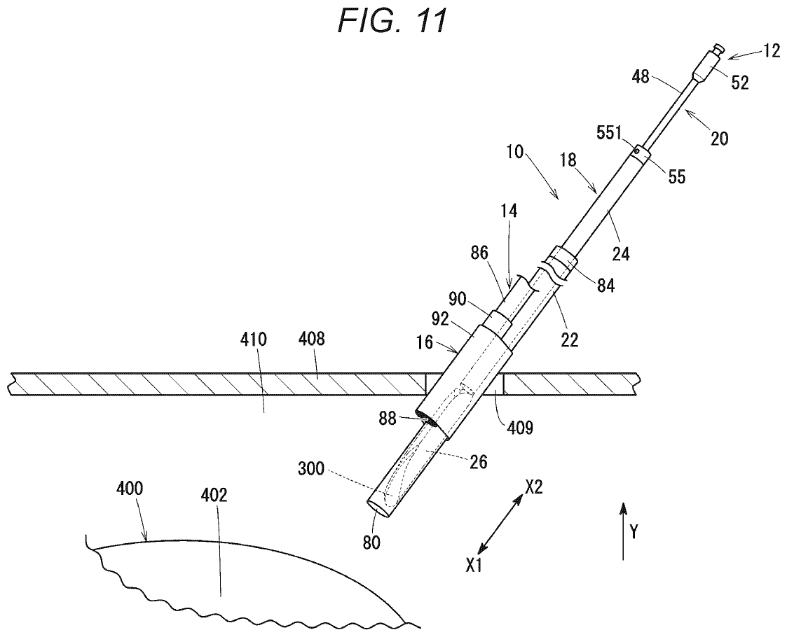

Subsequently, in the positioning process (step S4 in FIG. 6), as illustrated in FIG. 11, the transfer instrument 10 is inserted into a chest cavity 410 through an incision 409 in a chest 408. At this time, the distal end of the transfer instrument 10 is positioned near the recipient site 402 of the heart 400, and the distal end of the endoscope 14 is positioned in the chest cavity 410. Note that before the transfer instrument 10 is inserted into the chest cavity 410, a liquid supply instrument (not illustrated) may be connected to a connection port of the hub 52 to introduce a liquid (for example, a physiological saline solution).

Subsequently, in the unfolding process (step S5 in FIG. 6), as illustrated in FIG. 12, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 80 of the outer cylinder 22 returns to the original shape due to its restoring force. At the second position where the first support portion 26 is unfolded, the second support portion 50 unrolls flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 6), as illustrated in FIGS. 13 to 15, the second carrier member 20 is moved in the distal end direction (the direction of arrow X1) relative to the first carrier member 18. This causes the second support portion 50 on which the medical sheet 300 is placed to move from the retracted position to an advanced position and protrude in the distal end direction (the direction of arrow X1) relative to the distal end of the first support portion 26.

Specifically, in the moving process, the second shaft 48 is moved in the distal end direction relative to the first shaft 24. Accordingly, the second support portion 50 moves in the distal end direction (the direction of arrow X1) relative to the first support portion 26. At this time, the proximal end 304 of the medical sheet 300 enters the first recess 104 of the carrier holding portion 54 to come into contact with the pressing surface 58 (see FIGS. 13 and 14).

When the second shaft 48 is further moved in the distal end direction relative to the first shaft 24, the carrier holding portion 54 presses the proximal end 304 of the medical sheet 300 in the distal end direction via the pressing surface 58. Accordingly, the medical sheet 300 entirely moves in the distal end direction together with the second carrier member 20.

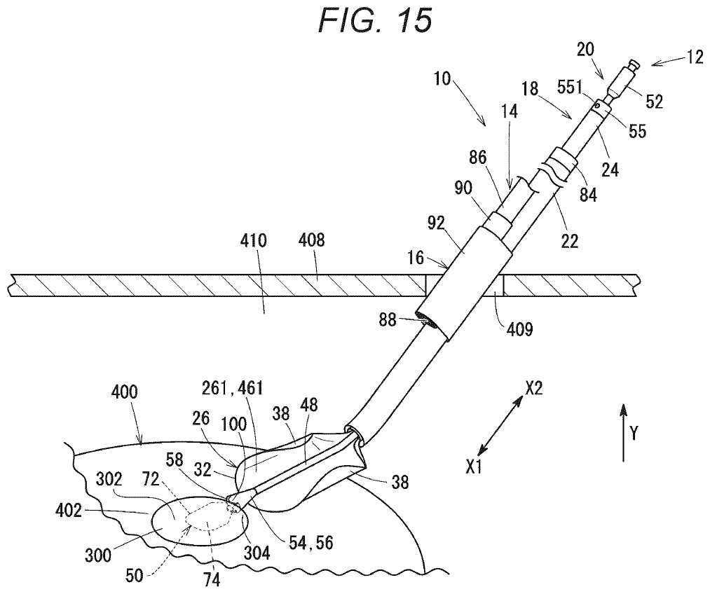

In this moving process, the medical sheet 300 is moved to above the recipient site 402 of the heart 400 to bring the overhanging portion 302 of the medical sheet 300 into contact with the recipient site 402 (see FIG. 15).

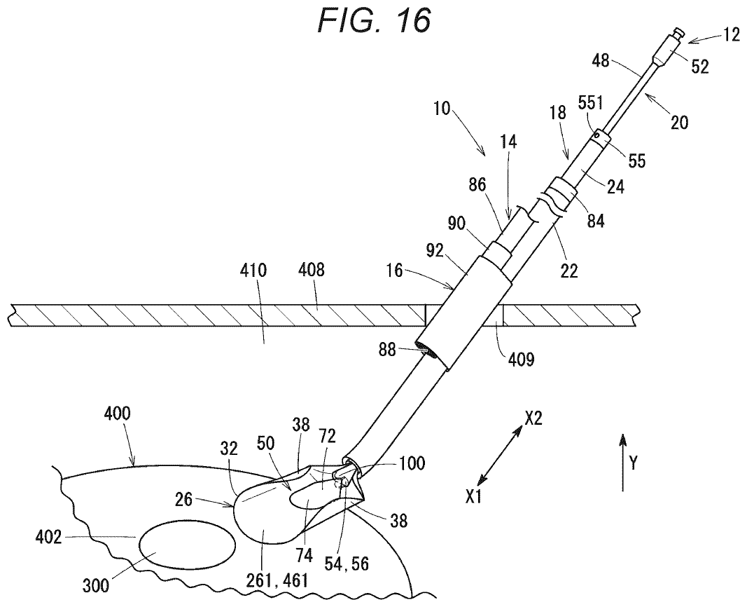

Subsequently, in the withdrawing process (step S7 in FIG. 6), as illustrated in FIG. 16, the second carrier member 20 is moved from the second position to the first position to withdraw the second support portion 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The first embodiment has the following effects.

As illustrated in FIG. 13, the second shaft 48 is moved in the distal end direction along the first shaft 24 with the medical sheet 300 supported on the first support surface 261. It is therefore possible to efficiently transfer the medical sheet 300 to the recipient site 402 of the heart 400 (the treatment site of the living body).

As illustrated in FIG. 4, in the carrier holding portion 54 (pressing portion), the first projection 100 (projection) protruding in the distal end direction relative to the pressing surface 58 is provided remote from the first support surface 261 relative to the pressing surface 58. Accordingly, as illustrated in FIGS. 13 and 14, when the second shaft 48 is moved in the distal end direction to cause the pressing surface 58 to press the medical sheet 300 in the distal end direction, the first projection 100 is positioned above the medical sheet 300, so that it is possible to prevent the medical sheet 300 from running on to the carrier holding portion 54. As a result, as illustrated in FIG. 15, the medical sheet 300 can be smoothly transferred to the recipient site 402 of the heart 400 (the treatment site of the living body).

As illustrated in FIGS. 4 and 5, the second support portion 50 is fixed to the pressing surface 58 of the carrier holding portion 54, and the medical sheet 300 is supported on the second support surface 74 of the second support portion 50. Accordingly, as illustrated in FIG. 14, when the second shaft 48 is moved in the distal end direction, the proximal end 304 of the medical sheet 300 enters the first recess 104 to come into contact with the pressing surface 58. As a result, as illustrated in FIG. 15, it is possible to smoothly transfer, by pressing the medical sheet 300 in the distal end direction with the pressing surface 58, the medical sheet 300 to the recipient site 402 while reliably preventing the medical sheet 300 from running on to the carrier holding portion 54.

As illustrated in FIGS. 4 and 5, the carrier holding portion 54 is provided with the second projection 102 (second projection) adjacent to the first support surface 261 of the first support portion 26 relative to the pressing surface 58. Accordingly, when the medical sheet 300 (see FIG. 1) is placed on the second support portion 50 with the second shaft 48 rotated by 180° about the axis of the second shaft 48, the second projection 102 is positioned above the medical sheet 300. At this time, even in a case where the second shaft 48 rotates about the axis of the second shaft 48, it is possible to cause, by moving the second shaft 48 in the distal end direction, the proximal end 304 of the medical sheet 300 to enter the second recess 106 to come into contact with the pressing surface 58. As a result, it is possible to smoothly transfer, by pressing the medical sheet 300 in the distal end direction with the pressing surface 58, the medical sheet 300 to the recipient site 402 while reliably preventing the medical sheet 300 from running on to the carrier holding portion 54.

As illustrated in FIG. 3, the first projection 100 has its distal end formed in an arc shape. It is therefore possible to prevent the medical sheet 300 (see FIG. 1) from breaking when the medical sheet 300 comes into contact with the distal end of the first projection 100.

In the first embodiment, the carrier holding portion 54 need not include the second projection 102.

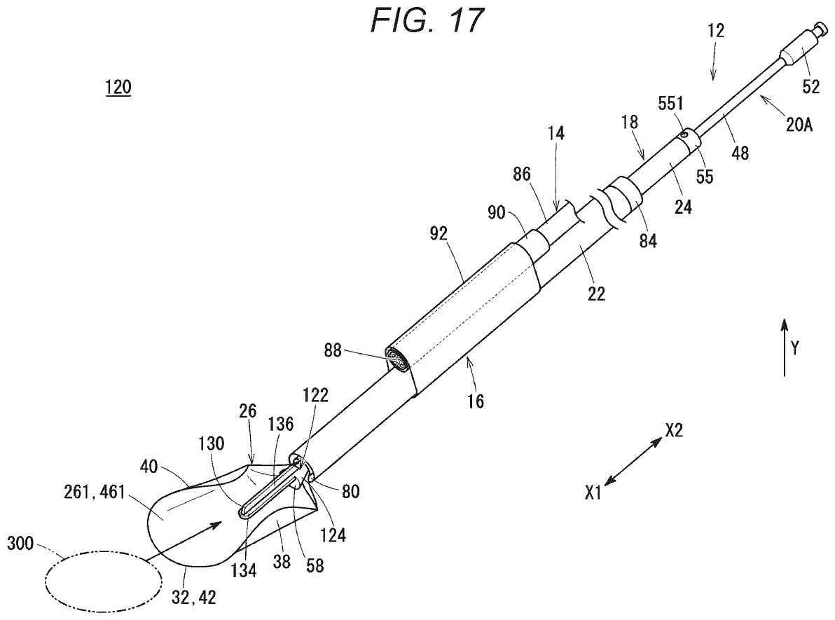

A transfer instrument 120 according to a second embodiment will be described with reference to FIGS. 17 to 23. Components of the transfer instrument 120 according to the second embodiment that are the same as of the transfer instrument 10 (see FIGS. 1 to 16) according to the first embodiment are denoted by the same reference numerals, and a detailed description of such components will not be repeated.

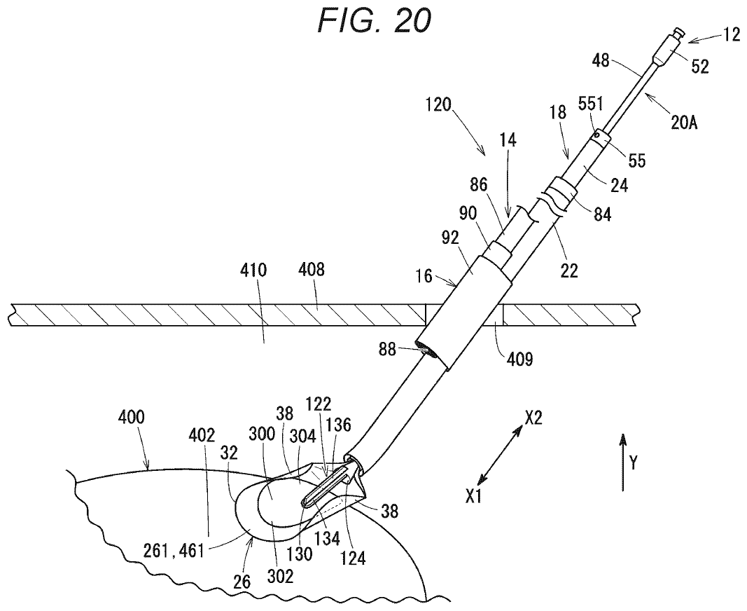

FIG. 17 is a perspective view of the transfer instrument 120 according to the second embodiment. The transfer instrument 120 does not include the second support portion 50 (see FIGS. 1 to 5). The medical sheet 300 is supported on the first support surface 261 of the first support portion 26 (see FIGS. 20 to 22).

That is, the transfer instrument 120 includes a second carrier member 20A. The second carrier member 20A includes the second shaft 48 and a pressing portion 122 provided at the distal end of the second shaft 48. The pressing portion 122 includes a pressing base portion 124 and a projection 130 protruding from the pressing base portion 124 in the distal end direction. The pressing base portion 124 has a distal end surface formed as the pressing surface 58. The projection 130 is provided at the distal end of the pressing base portion 124.

Specifically, as illustrated in FIG. 17, the projection 130 is provided remote from the first support surface 261 relative to the pressing surface 58 at the distal end of the pressing base portion 124. That is, the projection 130 is provided above the pressing surface 58 at the distal end of the pressing base portion 124. The projection 130 is provided at a center in the width direction of the distal end of the pressing base portion 124.

The projection 130 protrudes in the distal end direction relative to the pressing surface 58 at the distal end of the pressing base portion 124 and extends in the distal end direction. The projection 130 has a distal end formed in an arc shape. The shape of the distal end of the projection 130 is not limited to the arc shape. The distal end of the projection 130 may have any shape as long as the medical sheet 300 does not break when the distal end comes into contact with the medical sheet 300.

It is desirable that a protrusion length of the projection 130 from the distal end of the pressing base portion 124 in the distal end direction (protrusion length of the projection 130 in the distal end direction) be greater than or equal to half of the total length of the medical sheet 300 in the distal end direction. The protrusion length of the projection 130 is, for example, 10 mm to 50 mm. Therefore, when the medical sheet 300 is placed on the first support surface 261, the projection 130 is positioned above the medical sheet 300.

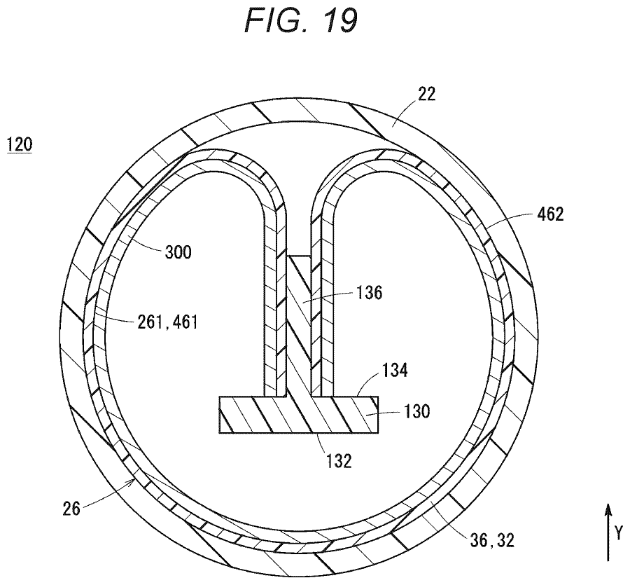

The projection 130 has a lower surface 132 facing the first support surface 261 and an upper surface 134 opposite to the lower surface 132. A rib 136 is provided on the upper surface 134 of the projection 130. The rib 136 is provided at a center of the upper surface 134 in the width direction. The rib 136 protrudes upward from the upper surface 134 of the projection 130 so as to be away from the first support surface 261. The rib 136 extends in the distal end direction on the upper surface 134 of the projection 130. Therefore, as illustrated in FIG. 19, the projection 130 and the rib 136 are formed in a T shape in a cross-sectional view. A proximal end of the rib 136 extends to an upper surface of the pressing base portion 124. It is therefore possible to prevent the projection 130 from hanging down toward the first support surface 261 under its own weight.

It is desirable that, in the transfer instrument 120, the pressing portion 122 include a flexible material. When the user pulls up the rib 136 using tweezers or the like, the projection 130 can be curved upward with a connection point with the pressing base portion 124 (proximal end of the projection 130) as a fulcrum.

Next, a transfer method for transferring the medical sheet 300 (see FIG. 17) in the second embodiment will be described.

In the preparing process (step S1 in FIG. 6), the transfer instrument 120 according to the second embodiment is prepared. For the transfer instrument 120, the state illustrated in FIG. 17 is the initial state.

In the sheet placing process (step S2 in FIG. 6), the medical sheet 300 placed in the Petri dish 401 (see FIG. 7) is placed on the first support surface 261. In the sheet placing process, the user pulls up the rib 136 using tweezers or the like. Accordingly, the projection 130 is curved upward with the connection point with the pressing base portion 124 (the proximal end of the projection 130) as a fulcrum. The user places the medical sheet 300 on the first support surface 261. In this case, the distal end of the projection 130 is separated upward from the first support surface 261 as compared with before the projection 130 is pulled up. It is therefore possible to place the medical sheet 300 on the first support surface 261 with the medical sheet 300 prevented from coming into contact with the distal end of the projection 130. After placing the medical sheet 300 on the first support surface 261, the user returns the projection 130 and the rib 136 to their respective original positions. Accordingly, the medical sheet 300 is placed (supported) between the first support surface 261 and the projection 130 on the first support surface 261.

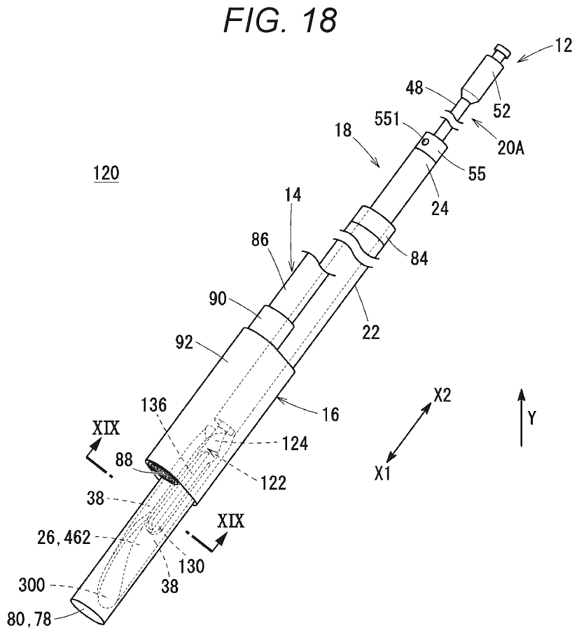

In the retracting process (step S3 in FIG. 6), as illustrated in FIG. 18, the medical sheet 300 is retracted in the outer cylinder 22 together with the first support portion 26 at the retracted position (first position). As illustrated in FIG. 19, the first support portion 26 is retracted in the outer cylinder 22 with the first support portion 26 curved in a heart shape along the inner surface of the outer cylinder 22. The medical sheet 300 is retracted in the outer cylinder 22 with the medical sheet 300 curved in a heart shape inside the first support portion 26. In the outer cylinder 22, the back surfaces 462 of both sides of the first support portion 26 in the width direction come into contact with the rib 136 while facing each other. In other words, in the outer cylinder 22, the rib 136 is sandwiched between the back surfaces 462 of both sides of the first support portion 26 in the width direction. Accordingly, the projection 130 is retracted in the outer cylinder 22 at the center of the outer cylinder 22 in the width direction.

In the positioning process (step S4 in FIG. 6), the transfer instrument 120 is inserted into the chest cavity 410 through the incision 409 in the chest 408.

In the unfolding process (step S5 in FIG. 6), as illustrated in FIG. 20, the first support portion 26 and the medical sheet 300 are unfolded with the medical sheet 300 supported by the first support surface 261. As described above, since the projection 130 is positioned at the center in the outer cylinder 22 (see FIG. 19), the first support portion 26 and the medical sheet 300 can be stably moved in the distal end direction and unfolded. When the first support portion 26 and the medical sheet 300 are unfolded, the proximal end 304 of the medical sheet 300 is positioned between the first support surface 261 and the projection 130.

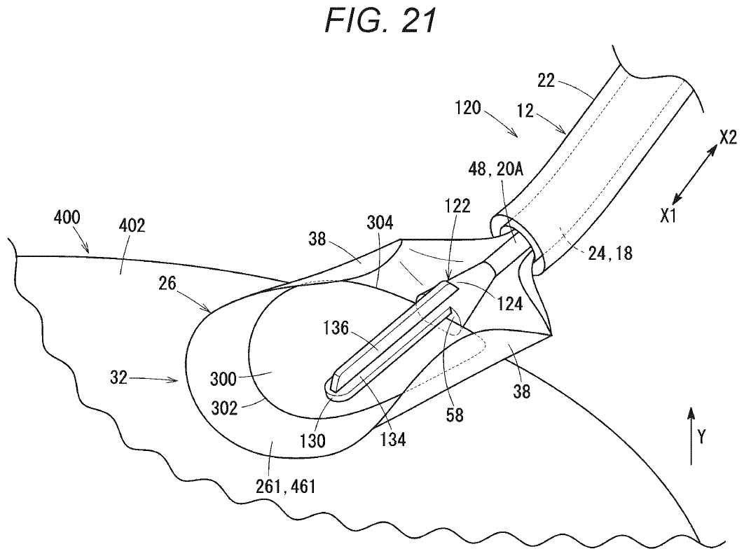

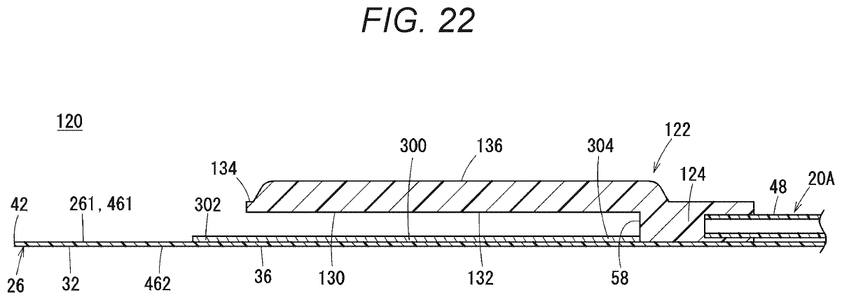

In the moving process (step S6 in FIG. 6), as illustrated in FIGS. 21 to 23, the second shaft 48 is moved in the distal end direction relative to the first shaft 24. Accordingly, the proximal end 304 of the medical sheet 300 comes into contact with the pressing surface 58 (see FIGS. 21 and 22).

When the second shaft 48 is further moved in the distal end direction relative to the first shaft 24, the pressing portion 122 presses the proximal end 304 of the medical sheet 300 in the distal end direction via the pressing surface 58. Accordingly, the medical sheet 300 entirely moves in the distal end direction together with the second carrier member 20A.

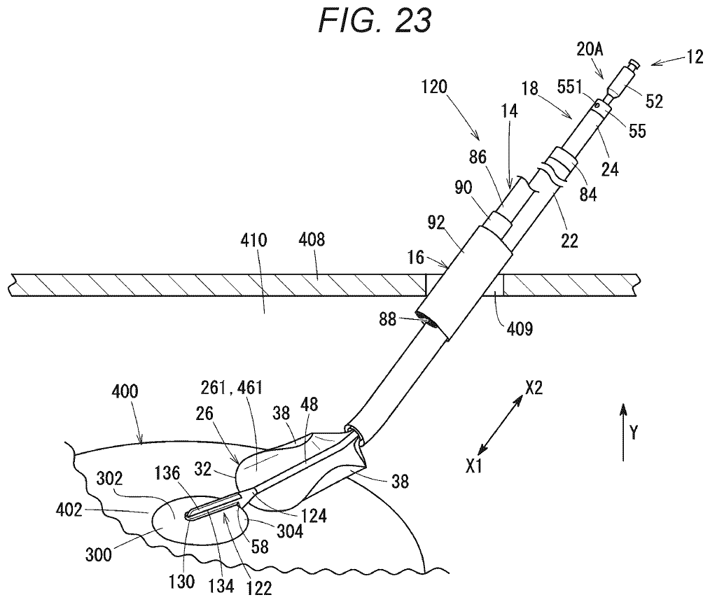

In the moving process, the medical sheet 300 is moved to above the recipient site 402 of the heart 400 to come into contact with the recipient site 402 (see FIG. 23). Accordingly, the medical sheet 300 entirely comes into contact with the surface of the recipient site 402. As a result, the transfer of the medical sheet 300 to the recipient site 402 is complete.

In the withdrawing process (step S7 in FIG. 6), the second carrier member 20A is moved from the second position to the first position. The transfer instrument 120 is withdrawn from the chest 408 with the first support portion 26 retracted in the outer cylinder 22.

The second embodiment has the following effects.

As illustrated in FIG. 17, in the pressing portion 122, the projection 130 (projection) protruding in the distal end direction relative to the pressing surface 58 is provided remote from the first support surface 261 relative to the pressing surface 58. That is, as shown, the projection 130 is spaced from the first support surface 261 such that the distance from the first support surface 261 to the projection 130 is greater than the distance from the first support surface 261 to the pressing surface 58. Accordingly, as illustrated in FIGS. 21 and 22, when the second shaft 48 is moved in the distal end direction to cause the pressing surface 58 to press the medical sheet 300 in the distal end direction, the projection 130 is positioned above the medical sheet 300, so that it is possible to prevent the medical sheet 300 from running on to the pressing portion 122. As a result, as illustrated in FIG. 23, the medical sheet 300 can be smoothly transferred to the recipient site 402 of the heart 400 (the treatment site of the living body).

As illustrated in FIGS. 21 and 22, since the protrusion length of the projection 130 in the distal end direction is greater than or equal to half of the total length of the medical sheet 300 in the distal end direction, the medical sheet 300 can be reliably supported between the first support portion 26 and the projection 130. Further, when the pressing surface 58 presses the medical sheet 300 in the distal end direction, it is possible to prevent the medical sheet 300 from becoming separated upward from the first support surface 261 and crinkled.

The pressing portion 122 with the projection 130 includes a flexible material (i.e., the pressing portion 122 with the projection 130 is made of a flexible material) and is provided with the rib 136 on the upper surface 134 of the projection 130. It is therefore possible to easily place the medical sheet 300 between the first support portion 26 and the projection 130 with the rib 136 lifted.

The projection 130 has a distal end formed in an arc shape. It is therefore possible to prevent the medical sheet 300 from breaking when the medical sheet 300 comes into contact with the distal end of the projection 130.

The present invention is not limited to the above-described disclosure, and various configurations can be adopted without departing from the gist of the present invention. That is, the detailed description above describes embodiments of a transfer instrument and transfer method for transferring a medical sheet to a treatment site of a living body, representing examples of the new transfer instrument and transfer method disclosed here. But the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A transfer instrument used to transfer a medical sheet to a treatment site of a living body, the transfer instrument comprising:
a first carrier member including a first shaft and a first support portion, the first support portion being provided at a distal end of the first shaft and having a first support surface configured to support the medical sheet;
a second carrier member including a second shaft and a pressing portion, the second shaft extending along the first shaft and being movable along the first shaft, the pressing portion being provided at a distal end of the second shaft; and
the pressing portion includes a pressing surface and a projection, the pressing surface being configured to move the medical sheet in a distal end direction when the medical sheet is supported on the first support surface, the projection being provided spaced from the first support surface relative to the pressing surface and protruding in the distal end direction relative to the pressing surface.

2. The transfer instrument according to claim 1, wherein
the second carrier member further includes a second support portion fixed to the pressing portion and having a second support surface smaller than the first support surface,
the medical sheet is supported on the second support surface during use of the transfer instrument, and
the pressing surface, the projection, and the second support portion together form a first recess that a proximal end of the medical sheet enters during use of the transfer instrument.

3. The transfer instrument according to claim 2, wherein
the pressing portion further includes a second projection provided adjacent to the first support surface relative to the pressing surface and protruding in the distal end direction relative to the pressing surface, and
the pressing surface, the second projection, and the second support portion form a second recess that the proximal end of the medical sheet enters during use of the transfer instrument.

4. The transfer instrument according to claim 1, wherein a protrusion length of the projection in the distal end direction is greater than or equal to half of a total length of the medical sheet in the distal end direction.

5. The transfer instrument according to claim 1, wherein
the pressing portion is made of a flexible material so that the pressing portion is flexible,
the projection includes a lower surface facing the first support surface and an upper surface opposite to the lower surface, and
on the upper surface, a rib protruding away from the first support surface and extending in the distal end direction is provided.

6. The transfer instrument according to claim 1, wherein the projection has an arc-shaped distal end.

7. A transfer instrument for transferring a medical sheet to a treatment site of a living body, the transfer instrument comprising:
an outer cylinder having a through hole that extends throughout the outer cylinder from a distal-end opening at a distal end of the outer cylinder to a proximal-end opening at a proximal end of the outer cylinder;
a first carrier member that includes a first shaft and a first support portion, the first shaft having a distal end portion at which is fixed the first support portion so that the first shaft and the first support portion move together, the first support portion extending in a distal direction away from the first shaft and having a first support surface configured to support the medical sheet, the first shaft being positionable in the outer cylinder so that a proximal portion of the first shaft is proximal of the proximal-end opening of the outer cylinder while the first support portion is positioned inside the outer cylinder, the first shaft being axially movable in a distal direction relative to the outer cylinder to move the first support portion from inside the outer cylinder to an extended position in which the first support portion is distal of the distal-end opening of the outer cylinder, the first shaft having a lumen that extends throughout the first shaft so that the lumen opens to a distal end of the first shaft and opens to a proximal end of the first shaft;
a second carrier member that includes a second shaft and a pressing portion, the second shaft having a distal end portion at which is fixed the pressing portion so that the second shaft and the pressing portion move together, the second shaft being positionable in the lumen of the first shaft with a proximal portion of the second shaft positioned proximal of the proximal end of the first shaft and the pressing portion positioned in the lumen of the first shaft;
the pressing portion having a pressing surface facing in the distal direction, the pressing portion including a projection that extends in the distal direction and that extends distally beyond the pressing surface, the projection having a lower surface; and
the second shaft, when positioned in the lumen of the first shaft, being axially movable in the distal direction relative to the first shaft when the first shaft is positioned in the outer cylinder with the first support portion in the extended position and the medical sheet supported on the first support portion so that the axial movement of the second shaft in the distal direction relative to the first shaft causes the pressing surface to contact a periphery of the medical device supported on the first support portion and move the medical device in the distal direction while the projection is positioned in overlying relation to the medical sheet and a lower surface of the projection faces toward and is spaced from the medical sheet.

8. The transfer instrument according to claim 7, wherein the projection has a distal end that faces in the distal direction away from the pressing surface, the distal end of the projection being curved.

9. The transfer instrument according to claim 7, wherein the projection is a first projection, and further comprising a second projection spaced apart from the first projection, the first projection being positioned in overlying relation to the second projection.

10. The transfer instrument according to claim 7, wherein the second carrier member includes a second support portion on which the medical sheet is positioned, the second support portion being fixed relative to the pressing portion and extending distally beyond a distal end of the first projection.

11. The transfer instrument according to claim 10, wherein the second support portion is smaller than the first support portion so that when the second support portion is positioned in overlying relation to the first support portion, the first support portion extends outwardly beyond an outer periphery of the second support portion.

12. The transfer instrument according to claim 10, wherein the second support portion, the second projection and a portion of the pressing surface between the second support portion and the second projection form a recess.

13. The transfer instrument according to claim 10, wherein the second support portion, the first projection and a portion of the pressing surface between the second support portion and the first projection form a recess that receives the periphery of the medical device supported on the first support portion.

14. The transfer instrument according to claim 7, wherein the projection is flexible and configured to be curved upwardly away from the first support portion in the extended position when grasped by tweezers held by a user.

15. The transfer instrument according to claim 7, wherein the projection includes an upper surface, and further comprising a rib on the upper surface of the projection, the projection having a width greater than a width of the rib.

16. The transfer instrument according to claim 15, wherein the projection has a distal end and the rib has a distal end, the distal end of the projection extending distally beyond the distal end of the rib.

17. A method comprising:
positioning an outer cylinder, a first carrier member and a second carrier member adjacent a site;
the first carrier member including a first shaft, a first support portion fixed to the first shaft and positioned in the outer cylinder, and a medical sheet surrounded by the first support portion,
the second carrier member including a second shaft and a pressing portion fixed to the second shaft and positioned in the outer cylinder, the pressing portion including a pressing surface and a projection;
axially moving the first shaft relative to the outer cylinder to move the first support portion to a position distal of a distal end of the outer cylinder so that the medical sheet is supported by the support portion and is positioned outside the outer cylinder;
axially moving the second shaft relative to the first shaft to move the pressing surface into contact with a periphery of the medical sheet; and
further axially moving the second shaft relative to the first shaft to move the pressing surface that is in contact with the periphery of the medical sheet in a distal direction to move the medical sheet in the distal direction relative to the first support portion.

18. The method according to claim 17, wherein the second carrier member includes a second support portion on which the medical sheet is positioned, the second support portion being fixed relative to the second shaft.

19. The method according to claim 17, wherein the projection overlies the medical sheet when the pressing surface is in contact with the periphery of the medical sheet.

20. The method according to claim 16, wherein a protrusion length of the projection in a distal end direction is greater than or equal to half of a total length of the medical sheet in the distal end direction.
